(19) United States
(12) Patent Application Publication
(10) Pub. No.: US 2022/0273641 A1
(43) Pub. Date: Sep. 1, 2022

(54) Method for treating coronavirus infections including SARS-CoV-2

(71) Applicants: Vera Mikhailov, Rolla, MO (US); Evgenii Ivanov, St. Petersburg (RU); Dmitrii Spelnikov, St. Petersburg (RU)

(72) Inventors: Vera Mikhailov, Rolla, MO (US); Evgenii Ivanov, St. Petersburg (RU); Dmitrii Spelnikov, St. Petersburg (RU)

(21) Appl. No.: 17/495,827

(22) Filed: Oct. 7, 2021

Related U.S. Application Data
(60) Provisional application No. 63/088,454, filed on Oct. 7, 2020.

(51) Int. Cl. A61K 31/4706; A61K 9/00; A61P 31/14; A61K 31/7052; A61K 31/675; A61K 31/7068

(52) U.S. Cl. CPC A61K 31/4706; A61K 9/0053; A61P 31/14; A61K 31/7052; A61K 31/675; A61K 31/7068

(57) ABSTRACT

The disclosure is directed to methods of using Floctafenine, or pharmaceutically acceptable salt of thereof, or derivative thereof, for treatment of patients with coronavirus infection including SARS-CoV-2. This compound is capable of inhibiting SARS-CoV-2 Main protease with highest specificity that allows the highest precision binding to the active site of SARS-CoV-2 Main protease. As a result of this targeted inhibition viral replication is expected to be blocked. That is expected to be a successful treatment of coronavirus infection including SARS-CoV-2. In addition to capability of inhibiting viral replication, Floctafenine is known for its anti inflammatory properties. That is expected to contribute to improving the outcome of subjects with inflammatory response as a result of coronavirus infection including SARS-CoV-2. Drug Repurposing is also expected to reduce healthcare costs given how laborious de novo drug discovery projects have been.

### FIELD OF THE INVENTION

The present invention relates to treating viral infection caused by a virus of the Coronaviridae family in subjects with SARS-CoV-2.

### BACKGROUND OF THE INVENTION

Coronaviruses are a group of related viruses that cause diseases in humans and animals. In humans, coronavirus is causing respiratory tract infections that are typically mild, such as some cases of the common cold (among other possible causes, predominantly coronaviruses), though rarer forms can be lethal, such as SARS, MERS, and COVID-19.

The emergence of the novel SARS coronavirus 2 (SARS-CoV-2) in 2019 has triggered an ongoing unprecedented global pandemic of highly infectious severe pneumonia-like disease designated as coronavirus disease 2019 (COVID-19). Newer variants of SARS-CoV-2 are still emerging. The number of confirmed cases worldwide continues to grow at a rapid rate, but, at this time, there are no antiviral drugs with proven clinical efficacy, nor are there universal COVID-19 vaccines available to control the symptoms or the spread of this disease caused by different variants of SARS-CoV-2 virus. The present invention is directed to overcoming these deficiencies in the art.

Both treatments and updated to most current variant vaccines will be needed to address the pandemic. The development of a universal vaccine is likely to require at least 12-18 months, and the typical timeline for approval of a novel antiviral therapeutic can exceed 10 years. Thus, repurposing of known drugs could significantly accelerate the deployment of novel therapies for COVID-19. The economic impact of COVID-19 is enormous and is difficult to estimate precisely. Drug repurposing can also reduce healthcare costs given how laborious, time consuming and costly de novo drug discovery projects have been.

### BRIEF DESCRIPTION OF THE DRAWINGS

FIG. 1 discloses essential role of SARS coronavirus main protease and replicase proteins in process of replication of virions triggering severe acute respiratory syndrome coronavirus (SARS-CoV) infections.

FIG. 2 shows how targeted inhibition of SARS-CoV-2 main protease results in blocking of replication of virus that can result in successful treatment of coronavirus infection in affected individuals.

FIG. 3 shows chemical structure of Floctafenine.

FIG. 4 discloses binding mode of Floctafenine to active center of SARS-CoV-2 main protease.

FIG. 5 shows magnified view of binding site of SARS-CoV-2 Main protease in complex with Floctafenine.

FIG. 6 shows a depiction of improved survival outcome using Floctafenine in combination with antiviral therapy.

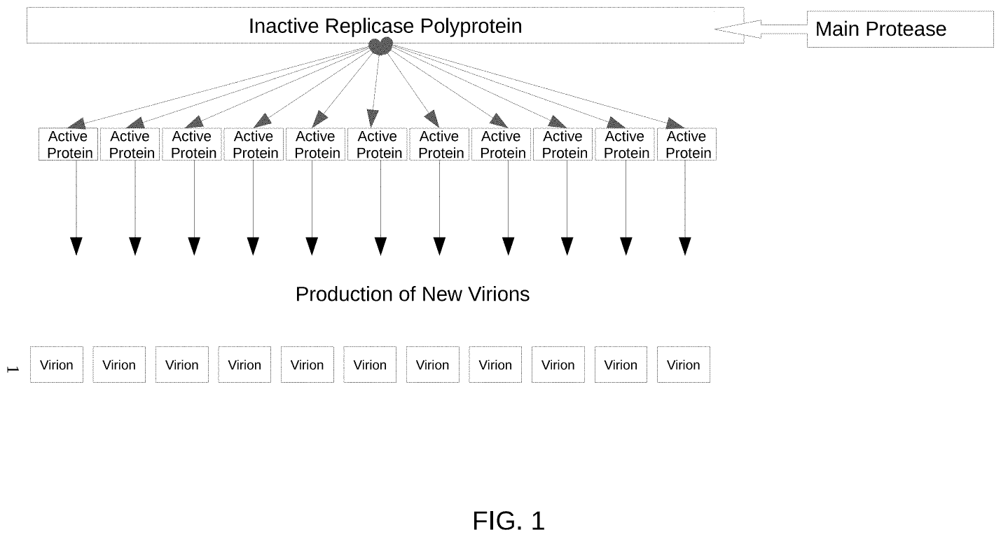

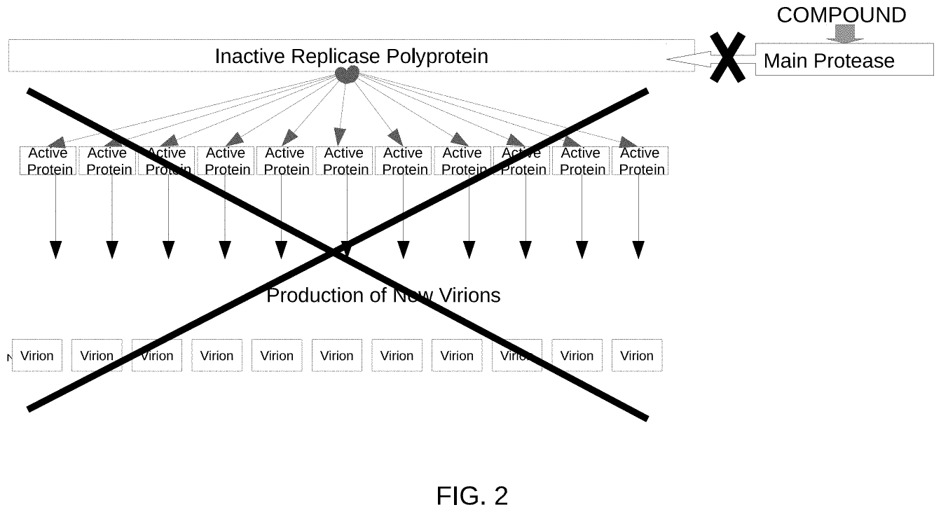

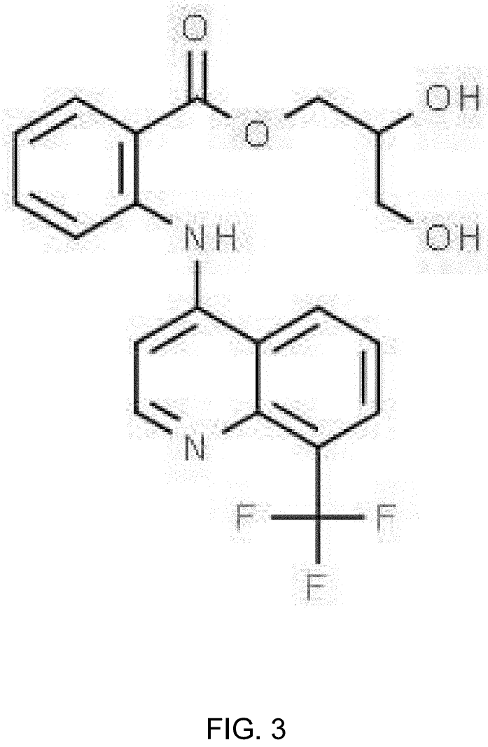

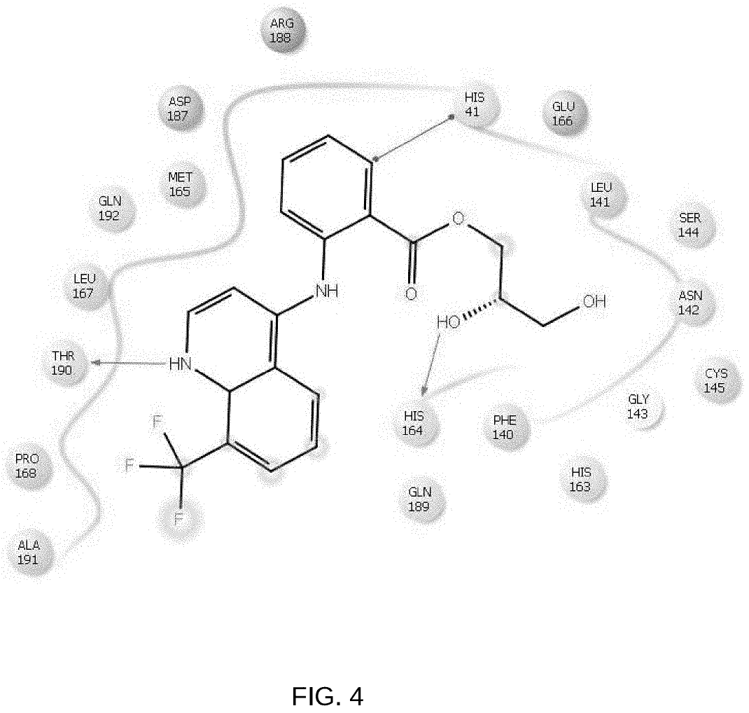

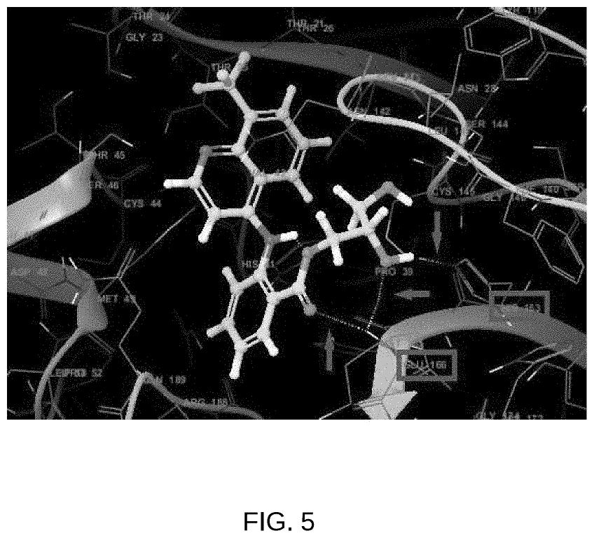

### DETAILED DESCRIPTION OF THE INVENTION

The presentation relates to a method of treating COVID-19 SARS 2 in a subject. This method includes selecting a subject with COVID-19 and administering to the selected subject a Compound A under conditions effective to treat the subject for COVID-19.

Aspects of the present specification disclose a method of treating an individual with viral infection including, without limitation, a viral (SARS-CoV-2) infection caused by a virus of the Coronaviridae family. The method comprises the step of administering to an individual in need thereof a pharmaceutical composition containing a therapeutically effective amount of Floctafenine or a pharmaceutically acceptable salt thereof which comprises administration of a therapeutic compound to treat viral infection, such as viral infection caused by a virus of the Coronaviridae family (SARS-CoV-2 infection) and to treat an inflammation. Aspects of the present specification further disclose a pharmaceutical composition comprising a therapeutic compound for treating an infectious disease, including, without limitation, SARS-CoV-2 and for treating a disorder associated with inflammation, wherein the pharmaceutical composition reduces symptoms of infection, including, without limitation, an infectious disease associated with viral infection caused by a virus of Coronaviridae family (SARS-CoV-2) and symptoms of inflammation including, without limitation, a systemic inflammatory response in the subject. Aspects of the present specification disclose treatment that can result in improvement of outcomes for many patients.

In one aspect, a method of preventing or reducing the risk of severe infection in a subject caused by exposure to a coronavirus is provided, the method comprising administering compound to the subject, systemically—via either oral or parenteral route. Parenteral route includes intramuscular, intravenous, subcutaneous injection. Endotracheal route is also considered.

According to the second aspect of the invention, there is provided the use of an agent capable of inhibiting viral replication, particularly by preventing Main protease from breaking down replicase polyprotein that occurs via proteolytic processing; for the treatment of infection caused by a virus from Coronaviridae family and reduction of inflammation, particularly systemic inflammatory response in a subject.

According to a third aspect of the invention, that is provided a composition comprising an agent capable of inhibiting SARS-CoV-2 Main protease, particularly by direct binding to the active site of SARS-CoV-2 Main protease resulting in prevention of proteolytic processing of inactive replicase; and a suitable pharmaceutical excipient.

According to a fourth aspect of the incident invention, that is provided a viral infectious disease treatment composition comprising an agent capable of penetrating the bilipid membrane of the host cell due to SARS-CoV-2 Main protease being a viable target inside the cell only and a suitable pharmaceutical excipient.

According to a fifth aspect of the invention, that is provided an agent with best bilipid membrane permeability capable of penetrating the bilipid membrane of the host cell due to SARS-CoV-2 Main protease being a viable target inside the cell only; and a suitable pharmaceutical excipient.

According to a sixth aspect of the incident invention, that is provided a viral infectious disease treatment composition comprising an agent with best binding energy to the active site of SARS-CoV-2 Main protease; and a suitable pharmaceutical excipient.

According to a seventh aspect of the incident invention, that is provided a viral infectious disease treatment composition comprising an agent with highest specificity that allows highest precision binding to the active site of SARS-CoV-2 Main protease; and a suitable pharmaceutical excipient.

### SUMMARY OF THE INVENTION

It is expected that the clinical use of Floctafenine for treating COVID-19 will improve the lives of many patients. Moreover, currently available treatments do not markedly improve outcome in severely ill patients. Also, the treatment of COVID-19, in accordance with the present invention, would provide a new alternative to antivirals. Because currently no antiviral drugs with proven clinical efficacy are available, it is possible that Floctafenine will provide a more efficacious antiviral alternative. The potential impact of Floctafenine is broad, because: a) COVID-19 is very widespread, b) COVID-19 virus is mutating and currently available vaccines may not provide effective protection against emerging newer variants of COVID-19 virus and c) illness caused by COVID-19 virus is associated with serious impairments including death.

In the summary, SARS-CoV-2 main protease can be inhibited by Floctafenine, that is bounding to active site of SARS-CoV-2 Main protease.

What is claimed is:

1. A method for the treatment of the 2019 coronavirus disease (COVID-19) caused by the SARS-CoV-2 virus in a subject including human in need of such treatment, the method comprising orally administering an effective amount of Floctafenine or a pharmaceutically acceptable salt of thereof, optionally in a pharmaceutically acceptable carrier, to the subject.

2. The method of claim 1, wherein the infection is caused by a virus from the group consisting of Coronaviridae including Severe Acute Respiratory Syndrome SARS-CoV.

3. The method of claim 1, where infection is caused by SARS-CoV-2 virus.

4. The method of claim 1, wherein infection is caused by variants of SARS-CoV-2 virus emerged as a result of mutation of the SARS-CoV-2 virus.

5. A method for treating a subject having COVID-19 infection and is in need of such treatment, the method comprising of orally administering effective amount of Floctafenine or pharmaceutically acceptable salt of thereof, to the subject so as to treat COVID-19 viral infection with variety of different presenting symptoms.

6. The method of claim 1, wherein the disease occurs within about one day from the start of the administration of the Floctafenine or a pharmaceutically acceptable salt of thereof.

7. The method of claim 1, wherein the disease occurs within about one week, or about two weeks, or about three weeks from the start of administration of Floctafenine or a pharmaceutically acceptable salt thereof.

8. The method of claim 1, wherein the method further comprises administering a second therapeutic agent.

9. The method of claim 8, wherein the second therapeutic agent is selected from the group consisting of the group Azithromycin, Remdesivir, Molnupiravir.

10. The method of claim 1 for the treatment of the 2019 coronavirus disease (COVID-19) caused by the SARS-CoV-2 virus in a subject in need thereof including human comprising administering an effective amount of Floctafenine or a pharmaceutically acceptable salt thereof, optionally in a pharmaceutically acceptable carrier, wherein the compound is administered according to the following schedule: A dose of 200 mg of Floctafenine every 12 hours or twice a day for 3 days.

11. The method of claim 1 for the treatment of the 2019 coronavirus disease (COVID-19) caused by the SARS-CoV-2 virus in a subject in need thereof including human comprising administering an effective amount of Floctafenine or a pharmaceutically acceptable salt thereof, optionally in a pharmaceutically acceptable carrier, wherein the compound is administered according to the following schedule: A dose of 200 mg of Floctafenine every 12 hours or twice a day for 4 days.

12. The method of claim 1 for the treatment of the 2019 coronavirus disease (COVID-19) caused by the SARS-CoV-2 virus in a subject in need thereof including human comprising administering an effective amount of Floctafenine or a pharmaceutically acceptable salt thereof, optionally in a pharmaceutically acceptable carrier, wherein the compound is administered according to the following schedule: A dose of 200 mg of Floctafenine every 12 hours or twice a day for 5 days.
